(19) United States
(12) Plant Patent Application Publication
(10) Pub. No.: US 2007/0271652 P1
(43) Pub. Date: Nov. 22, 2007

(54) IMPATIENS PLANT NAMED 'IMDOSAOR'

(75) Inventor: Monica Maria Adelheid Sanders, Grootebroek (NL)

Correspondence Address: SYNGENTA BIOTECHNOLOGY, INC.;PATENT DEPARTMENT, 3054 CORNWALLIS ROAD, P.O. BOX 12257, RESEARCH TRIANGLE PARK, NC, 27709-2257, US

(73) Assignee: Syngenta Seeds B.V.

(21) Appl. No.: 11/438,044

(22) Filed: May 19, 2006

(51) Int. Cl. A01H 5/00

(52) U.S. Cl. PLT/317

(57) ABSTRACT

A new Impatiens plant, characterised particularly as to novelty by an upright habit, freely branching, with large, double, salmon orange flowers, flowering above the foliage.

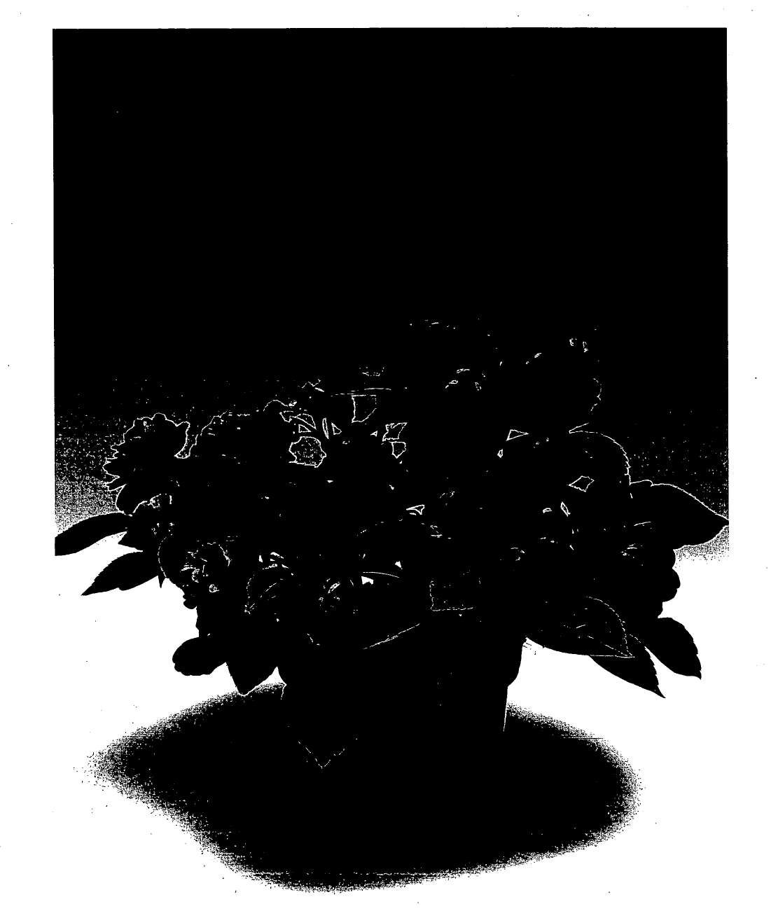

### LATIN NAME OF THE GENUS AND SPECIES OF THE PLANT CLAIMED

Impatiens walleriana

### VARIETAL DENOMINATION

‘Imdosaor’

### BACKGROUND OF THE NEW PLANT

The present invention comprises a new distinct cultivar of Impatiens, botanically known as Impatiens walleriana.

The new Impatiens is a product of a planned breeding program conducted in Enkhuizen, Netherlands. The new Impatiens cultivar is upright growing, early flowering, floriferous with large, double flowers and has attractive foilage.

The present cultivar originated from a pollination made in 2001, of a proprietary Impatiens selection identified as ‘S3244-1,’ not patented, as the female, or seed parent, with a proprietary Impatiens selection identified as ‘S3163-1,’ not patented, as the male, or pollen parent.

The present cultivar has been repeatedly asexually reproduced by cuttings in Enkhuizen, Netherlands, in Angers, France, in Gilroy, USA over a four year period. The distinctive characters of this new Impatiens are stable and reproduced true to type in successive generations of asexual reproduction. It takes 6 to 8 weeks to produce a finished plant, depending on the temperature.

This new Impatiens plant is an annual in most climatical zones in the US, only in zones 9 and 10 it is a perennial plant.

### DESCRIPTION OF THE DRAWING

This new Impatiens plant is illustrated by the accompanying photographic drawing which shows blooms, buds and foliage of the plant in full color, the color showing being as true as can be reasonably obtained by conventional photographic procedures.

### DESCRIPTION OF THE NEW CULTIVAR

The following detailed descriptions set forth the distinctive characteristics of this new Impatiens. The data which defines these characteristics were collected from asexual reproductions carried out in Enkhuizen, Netherlands. The plant history was taken on 25 week old plants, blossomed under natural light in the field.

Color readings were taken in the office under ambient light. Color references are primarily to the R.H.S. Color Chart of The Royal Horticultural Society of London.

Plants of the new Impatiens differ primarily from the plants of the female parent selection in the following characteristics:

Plants of the new Impatiens have orange, double flowers and flowers only have petals, whereas the plants of the female have orange flowers and flowers have a pollen cap and stigma.

Plants of the new Impatiens differ primarily from the male plant selection in the following characteristics:

Plants of the new Impatiens have orange colored flowers, whereas the plants of the male have salmon colored flowers.

TABLE 1
DIFFERENCES BETWEEN THE NEW CULTIVAR ‘IMDOSAOR’
AND A SIMILAR CULTIVAR

| | | ‘Salmon Sunrise’ |
|---|---|---|
| | | (U.S. Plant Patent |
| | ‘Imdosaor’ | No. 9,691) |
| Leaf color | Dark green | Medium green |
| Flower size | Large | Smaller |
| Number of petals | Very high number | Normal number |

PARENTAGE

| | |
|---|---|
| Female parent: | ‘S3244-1,’ not patented |
| Male parent: | ‘S3163-1,’ not patented |

PROPAGATION

| | |
|---|---|
| Type cutting: | Terminal cuttings |
| Time to initiate roots: | 10-12 days at 20 degrees C. |
| Time to produce a | 4 weeks |
| rooted cutting: | |
| Root description: | Fine, fibrous and white in color |

PLANT DESCRIPTION

| | |
|---|---|
| General appearance: | Compact, mounded and outwardly |
| | spreading. Appropriate for 12 |
| | cm pots and large containers |
| Growth and | Upright growing plant habit, well |
| branching habit: | branched |
| Crop time: | 6-8 weeks, starting from an unrooted |
| | cutting in a 10.5 cm pot |
| Plant height: | 12 cm |
| Plant diameter or spread: | 44.5 cm |

LATERAL BRANCHES

| | |
|---|---|
| Length: | About 21 cm |
| Diameter: | About 5 mm |
| Internode length: | About 2 cm |
| Texture: | Smooth |
| Color: | 146D |
| Anthocyanin may be present: | 59B |

FOLIAGE DESCRIPTION

| | |
|---|---|
| Arrangement: | Simple, alternate |
| Length: | About 4.6 cm |
| Width: | About 2.7 cm |
| Shape: | Ovate |
| Apex: | Acuminate |
| Base: | Attenuate |
| Margin: | Serrate, rounded |
| Texture: | Glabrous, smooth |
| Venation pattern: | Pinnate, arcuate |
| Color, developing and | 137A |
| fully expanded foliage, | |
| upper surface: | |
| Color, developing and | 147C |
| fully expanded foliage, | |
| lower surface: | |
| Venation, upper and | 137C |
| lower surfaces: | |
| Petiole, length: | About 2.1 cm |
| Petiole, diameter: | About 2 mm |
| Color: | 49D with green |

FLOWER DESCRIPTION

| | |
|---|---|
| Flower type and | Numerous and consistently large fully |
| flowering habit: | double flowers. Freely and |
| | continuously flowering. Flower buds |
| | open similar to a rose in fullness; |
| | flowers rounded. Flowers arise from |
| | leaf axils. Typically about 24 |
| | flowers and flower buds per lateral |
| | branch. Flowers positioned above |
| | and beyond the foliage and typically |
| | face outward. Flowers last about five |
| | to seven days under greenhouse |
| | conditions. Flowers not persistent. |
| | Flowers not fragrant. |
| Flowering season: | Year round under greenhouse conditions. |
| | In the garden, flowering is continuous |
| | from Spring until Fall. |

Flower size

| | |
|---|---|
| Diameter: | About 4 cm |
| Depth: | About 2 cm |

FLOWER BUDS

| | |
|---|---|
| Length, at stage for | About 1.2 cm |
| showing color: | |
| Diameter, at stage | About 10 mm |
| for showing color: | |
| Shape: | Ovoid |
| Color: | 55D |

PETALS

| | |
|---|---|
| Quantity/arrangement: | About 40 per flower; imbricate |
| Length, outer petals: | About 2.4 cm |
| Width, outer petals: | About 1.9 cm |
| Shape: | Oval to obovate |
| Apex: | Emarginate to retuse |
| Base: | Shortly attenuate |
| Margin: | Entire |
| Texture, upper and lower | Velvety, smooth |
| surfaces: | |

Color

| | |
|---|---|
| When opening, upper | 40A |
| surface: | |
| When opening, lower | 43D |
| surface: | |
| Fully opened, upper | 40A |
| surface: | |
| Fully opened, lower | 43D |
| surface: | |

SPURS

| | |
|---|---|
| Length: | About 2.3 cm |
| Color: | N144D |

SEPALS

| | |
|---|---|
| Quantity/arrangement: | Five in a single whorl |
| Calyx length: | About 3 mm |
| Calyx diameter: | About 2 mm |
| Shape: | Elliptic |
| Apex: | Acute |
| Margin: | Entire |
| Texture, upper and | Smooth |
| lower surfaces: | |

Color

| | |
|---|---|
| Upper surface: | 145A |
| Lower surface: | 145A |

PEDUNCLES

| | |
|---|---|
| Length: | About 2.1 cm |
| Diameter: | About 2 mm |
| Angle: | About 45 degrees to the |
| | lateral branch |
| Strength: | Moderately strong |
| Color: | 152C |

REPRODUCTIVE ORGANS

None observed

SEED/FRUIT DEVELOPMENT

Seed and fruit development has not been observed

What is claimed is:

1. A new and distinct cultivar of Impatiens plant named ‘Imdosaor,’ as substantially illustrated and described herein.
